(19) United States
(12) Patent Application Publication
(10) Pub. No.: US 2019/0262126 A1
(43) Pub. Date: Aug. 29, 2019

(54) ARTIFICIAL ACCOMMODATING LENS COMPLEX

(71) Applicants: Arthur Bernard CUMMINGS, Dublin (IE); Francis BOLLE, Johannesburg (ZA)

(72) Inventor: Francis Bolle, Johannesburg (ZA)

(21) Appl. No.: 16/318,953

(22) PCT Filed: Jul. 12, 2017
(86) PCT No.: PCT/EP2017/067617

§ 371 Date: Jan. 18, 2019

(30) Foreign Application Priority Data
Jul. 19, 2016 (DE) 10 2016 008 793.5

(51) Int. Cl. A61F 2/16

(52) U.S. Cl. CPC A61F 2/1635; A61F 2002/16901

(57) ABSTRACT

The present invention provides an arrangement (600) for an artificial eye lens. The arrangement comprises at least two ring-shaped elements (610) and a plurality of arched ribs (620). The plurality of arched ribs connects the two ring-shaped elements. The ring-shaped elements and plurality of arched ribs form a space for housing the artificial eye lens. The ring-shaped elements arc moveable with respect to each other along a common axis (x). It comprises further a collapsible tape (64) for define a maximum range of deformation of the arrangement.

The present invention generally relates to the field of artificial intraocular lenses. More particularly, the present invention relates to an arrangement for an artificial eye lens and to a surgical method using the arrangement.

An artificial eye lens, also often called an intraocular lens (IOL), is usually a lens implant in the eye used to treat certain defects of the eye such as cataract or refractive error (myopia, hyperopia or astigmatism). IOLs were traditionally made of an inflexible material, although this has largely been superseded by the use of flexible materials. Most IOLs used today are fixed monofocal lenses matched to a fixed distance vision. However, other types are available, such as multifocal IOLs which provide the patient with multi-focused vision at far and near distances and adaptive IOLs which provide the patient with limited visual accommodation. IOLs enable many patients to have reduced dependence on glasses. However, most patients still rely on glasses for certain activities, such as reading.

In more detail, the design of monofocal lenses means that they are, in general, only capable of providing vision correction at nearsightedness or farsightedness. Although it is possible to attempt to correct one eye for distance vision and one for near vision (monovision), monofocal lens recipients generally require reading glasses or bifocals for close reading vision after surgery.

Multifocal IOLs address this issue directly by offering a lens replacement solution that boasts a design capable of restoring vision across varying distances. Multifocal IOLs provide several different focusing distances within the same lens. On the contrary, accommodating IOLs normally have only one focusing distance in the lens but the lens actually allows the eye to change focusing distances as one looks at distant or near objects. Because there is only one focusing distance in the lens at any one time, there is no loss of quality of vision at any distance (unlike a multifocal). However, current accommodating or adaptive IOLs do not provide the same range of focus as young eyes, and may not allow one to see at very close distances without reading glasses, like one can with multifocal IOLs.

One of the major disadvantages of conventional IOLs is that they are primarily focused for distance vision. The patients who undergo a standard IOL implantation, no longer experience clouding from cataracts. However, they are unable to accommodate, or change focus from near to far, far to near, and to distances in between. One so-called accommodating or adaptive IOL interacts with ciliary muscles, zonules and capsule, using hinges at both ends to latch on and to move the optic forward and backward inside the eye.

So in summary, a drawback of the state of the art IOLs is that they do not accommodate like the natural human lens for far and near.

Therefore, it is an object of the present invention to provide an improved technique for accommodation of an artificial eye lens.

In a first aspect of the present invention an arrangement for an artificial eye lens is provided. The arrangement comprises at least two ring-shaped elements and a plurality of arched ribs. The plurality of arched ribs connects the two ring-shaped elements. The ring-shaped elements and the plurality of arched ribs form a space for housing the artificial eye lens. The ring-shaped elements are moveable, with respect to each other, along a common axis.

The artificial eye lens may be an accommodating artificial eye lens.

In this respect, accommodation may be understood as having an arrangement housing an artificial eye lens. Further, accommodation may be understood as this artificial eye lens adjusting or accommodating depending on the viewing distance of the human eye.

Generally, accommodation may be understood as a process that enables the eye to adjust its focusing power to provide clear vision at one or more, e.g. at all, distances.

Accommodation can be made possible by the lens inside the eye and the circular muscle that surrounds the lens, called the ciliary muscle. The lens and ciliary muscle are connected by a 360-degree series of fibers (called ciliary zonules) that extend from the ciliary muscle to the thin lens capsule (or “bag”) that encloses the lens. The ciliary muscle, ciliary zonules and lens capsule keep the lens suspended in its proper position inside the eye for clear vision and effect accommodation in the lens.

In a normal eye (without presbyopia or cataracts), this dynamic process of accommodation adjusts the focusing power of the eye by changing the thickness and curvature of the eye's natural lens. When the ciliary muscle is relaxed, there is tension on the ciliary zonules and lens capsule and the lens flattens to enable clear distance vision. When the ciliary muscle contracts, the ciliary zonules and lens capsule relax and the lens is allowed to thicken by its natural tendency to be more rounded, becoming more curved for added focusing for clear near vision. In a young eye, accommodation is essentially instantaneous and effortless. As the eye ages, the lens becomes less flexible, causing the loss of near vision that is the hallmark sign of presbyopia in people over age 40.

During cataract surgery, a circular opening is normally created in the anterior part of the lens capsule so the surgeon can remove the eye's natural lens contents that have become cloudy. This step is called an anterior capsulotomy. The peripheral and posterior portions of the lens capsule are left intact, forming a partially open “bag” that the intraocular lens is positioned within to restore focusing power to the eye. IOLs have a central optical zone, with peripheral “legs” (often called haptics) that secure the lens implant inside the lens capsule or bag. The primary difference between a conventional monofocal IOL and an accommodating IOL is the design of these haptics. In a conventional IOL, the haptics are designed to keep the optical portion of the implant stationary, with no rotation or anterior/posterior movement that could affect vision. In an accommodating IOL, the design becomes more complex.

The at least two ring-shaped elements can be soft and malleable. For example, the material of the at least two ring-shaped elements may be or comprise silicon, acrylic, gel or related material that does not expand circumferentially or only a little. The at least two ring-shaped elements may be deformed for implantation. The ribs of the plurality of arched ribs may be of the same material as the ring-shaped elements.

The common axis may be defined by or as an axis on which the at least two ring-shaped elements are aligned and/or along which the at least two ring-shaped elements are moveable.

The arrangement may further comprise the artificial lens/artificial eye lens.

The artificial lens can be soft and be deformed by the action of one or more components or parts of the arrangement.

The artificial eye lens may be made of soft gel, silicon, or any related useful biocompatible material or a lens comprising a capsule filled with fluid silicon or related material, so that it can be deformed.

The space being formed by the ring-shaped elements and the plurality of arched ribs may be understood as the interior of the arrangement.

The advantage of the arrangement is that the arrangement implanted in the capsular bag of an eye transfers the forces of natural human accommodation to the artificial lens suspended in the space within the arrangement. In this way, the optical portion of the lens can be deformed, flatter for distance vision and rounder for near vision. The artificial lens can be designed to be naturally more rounded at rest to allow for this action.

The ring-shaped elements may be arranged in parallel to each other. The arched ribs may be flexible and/or stretchable. The ring-shaped elements may further be arranged coaxially.

The arrangement may comprise haptics that are configured and arranged to allow the artificial eye lens to be connected thereto within the space.

The haptics provide the advantage of enabling a small artificial lens to be suspended. The same constricting or expanding movements provided by the zonules lead to an amplified deformation of the artificial lens. Therefore, a large range of viewing distances can be accommodated by the arrangement comprising a smaller artificial lens than the natural human lens.

The haptics may comprise one or more legs or strands, respectively protruding from at least one of the plurality of arched ribs into the interior of the space. The one or more legs or strands may be arranged at a center or a vicinity of the center of each of the arched ribs. For example, the haptics may include peripheral legs that secure the lens implant to one or more of the plurality of arched ribs inside the lens capsule, more particularly in a central optical zone of the lens arrangement.

The ring-shaped elements may be moveable, with respect to each other, along the common axis at least between a first position and a second position. In the first position, the arched ribs may be more flattened than in the second position. In the first position, the distance between the two ring-shaped elements along the common axis may be larger than in the second position.

The arrangement may be configured to at least one of hold, move and deform the artificial eye lens. The arrangement may be adapted to adjust a distance focus of the artificial eye lens. Adjusting the distance focus may be achieved by deforming and/or moving the artificial eye lens. For example the arrangement may be adapted to adjust the distance focus of the artificial eye lens by deforming the artificial eye lens, or by moving the artificial eye lens along the common axis. Moving may further include deforming the artificial eye lens.

The arrangement may further comprise a collapsible tape. The collapsible tape may be adapted and arranged to link the arched ribs. The collapsible tape may be adapted and arranged to define a maximum range of deformation of the arrangement. The collapsible tape may be linked from any part to any other part of the arrangement or any arrangement. The collapsible tape may be a movement limiting device and may assume to be a plate, a string or a stop.

The second position may be set by an extent of the collapsible tape. The first position may be set by a force being the least force applied on the arrangement. The extent may be understood as the maximum deformation of the collapsible tape.

The second position may be set to a determined distance focus. This has the advantage of having a precise viewing capability for a desired viewing distance.

The collapsible tape may be arranged on the center or a vicinity of the center of each of the arched ribs.

In a second aspect, a collapsible tape is provided. The collapsible tape is adapted and arrangeable to link arched ribs. The collapsible tape is further adapted and arrangeable to define a maximum range of deformation of an arrangement according to the first aspect.

In another aspect, a surgical method is provided. The method comprises introducing an incision into the eye. The method further comprises introducing elements of the arrangement according to the first aspect into the interior of the eye. The method may further comprise at least partially assembling the arrangement in the eye. The arrangement may at least partially be assembled in the eye by using the elements introduced into the interior of the eye.

A small incision can be achieved by a small lens design. This has the advantage of minimizing a risk of affecting the cornea by astigmatism.

Even if some of the aspects described above have been described with respect to the arrangement, these aspects may also apply to the surgical method using the arrangement.

In the following, the present disclosure will be further described with reference to exemplary embodiments illustrated in the Figures, in which:

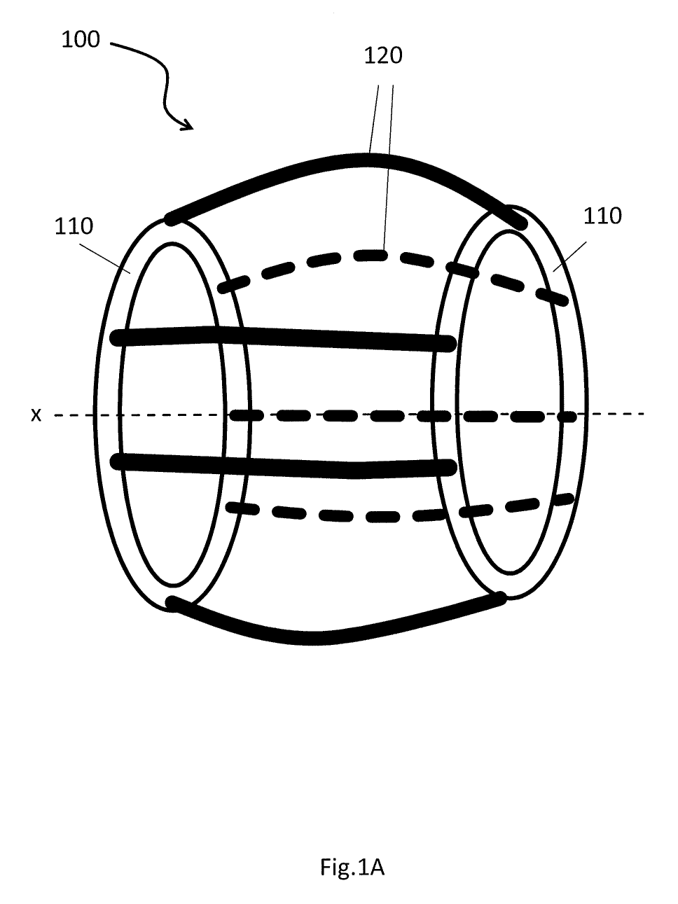

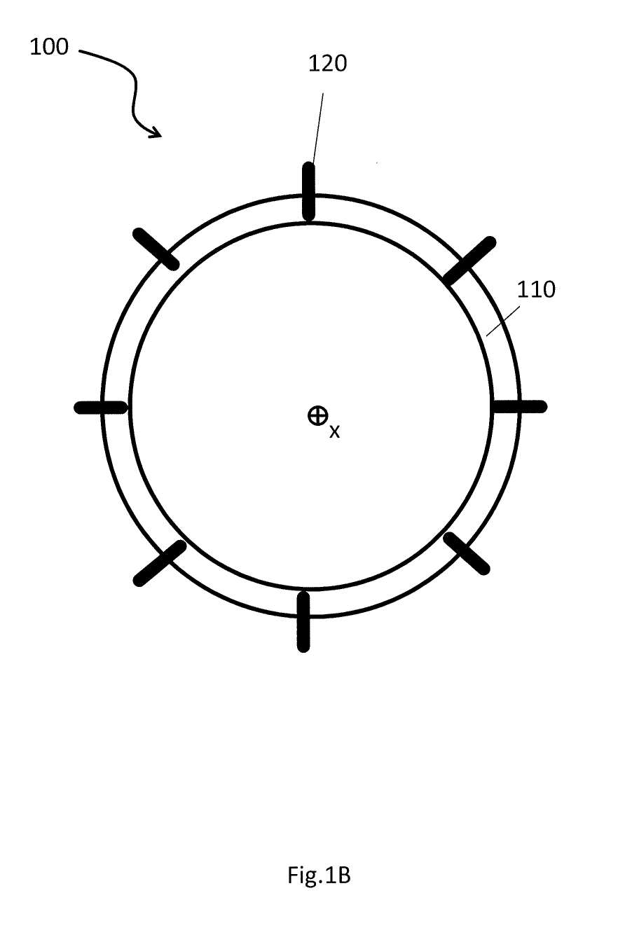

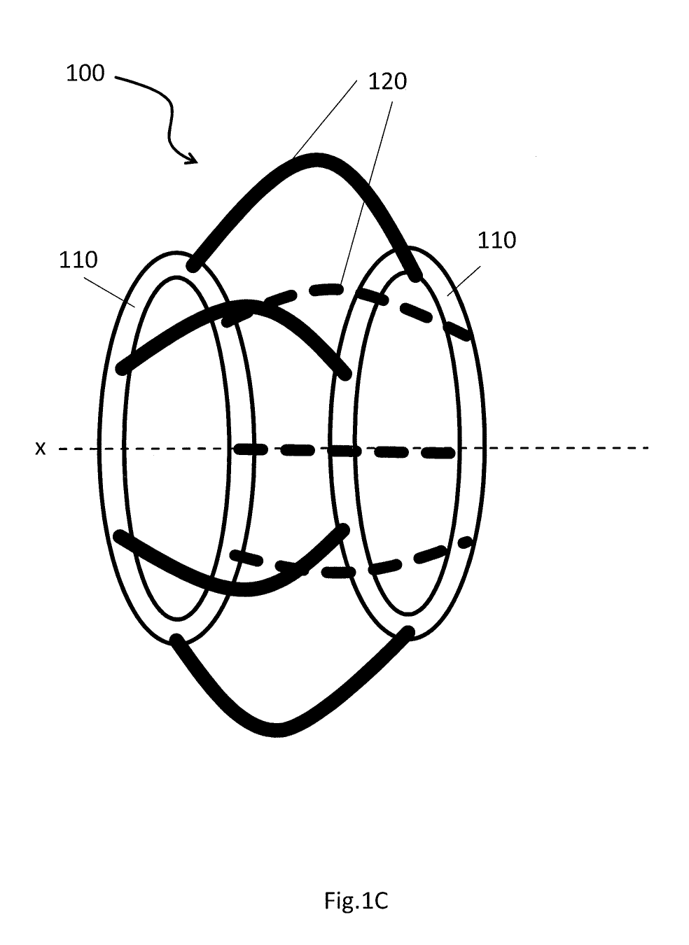

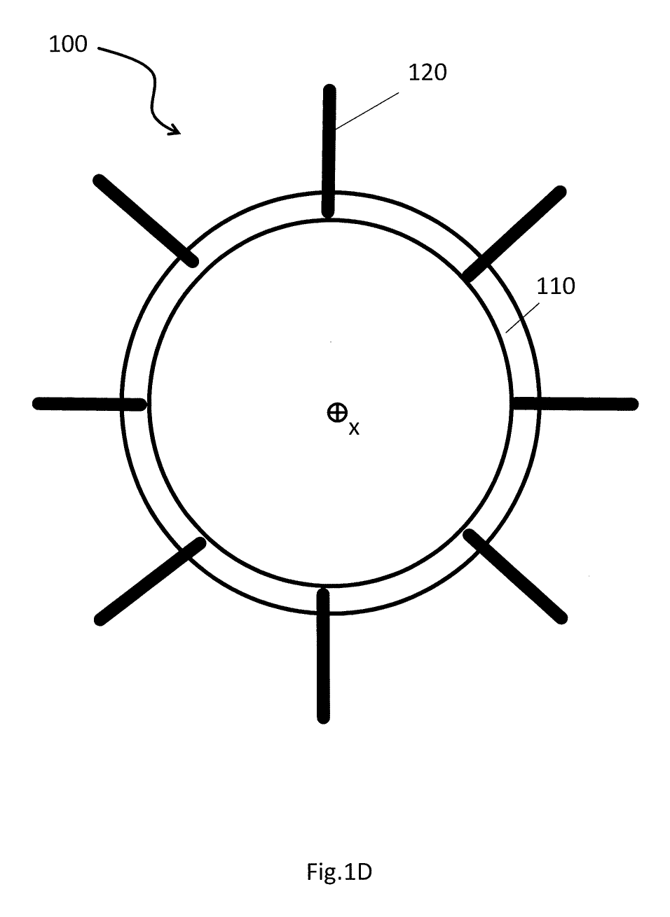

FIG. 1A schematically illustrates an arrangement according to an embodiment of the present invention in a side view;

FIG. 1B schematically illustrates an arrangement according to an embodiment of the present invention in a rear view;

FIG. 1C schematically illustrates an arrangement according to an embodiment of the present invention in a side view;

FIG. 1D schematically illustrates an arrangement according to an embodiment of the present invention in a rear view;

FIG. 2 schematically illustrates an arrangement according to an embodiment of the present invention with additional haptics in a rear view;

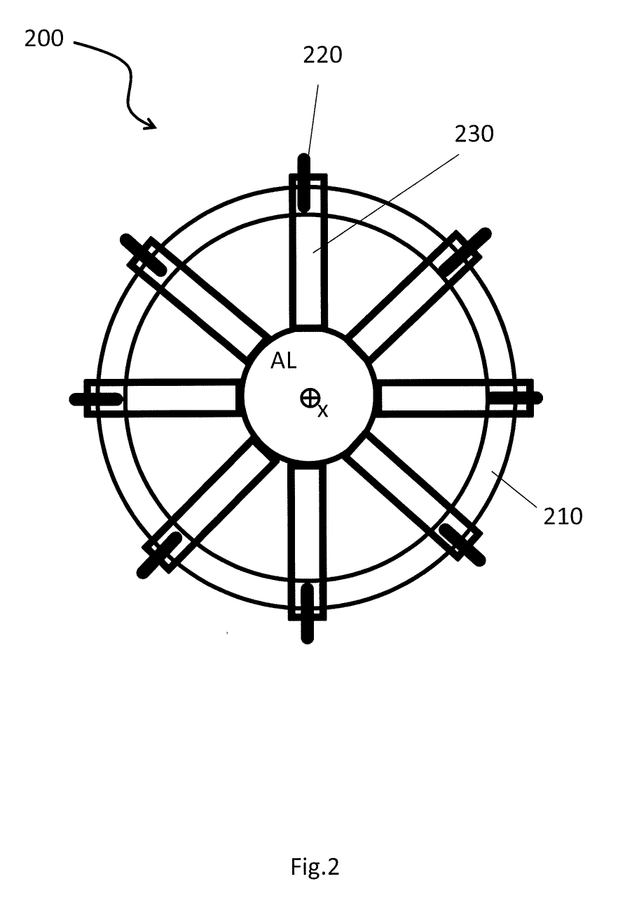

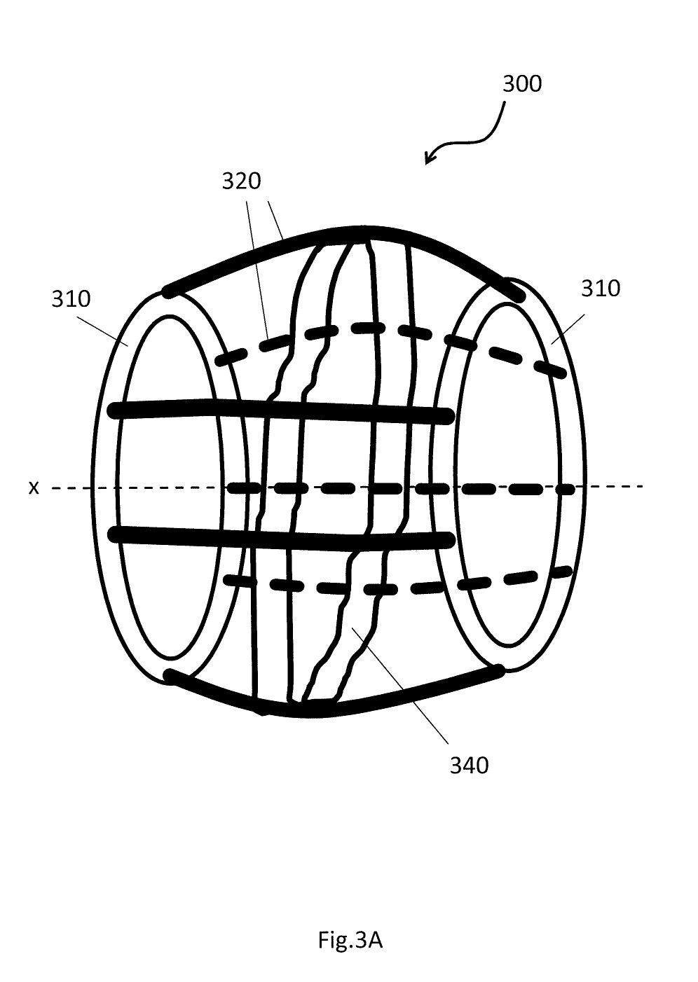

FIG. 3A schematically illustrates an arrangement according to an embodiment of the present invention with an additional collapsible tape in a side view;

FIG. 3B schematically illustrates an arrangement according to an embodiment of the present invention with an additional collapsible tape in a rear view;

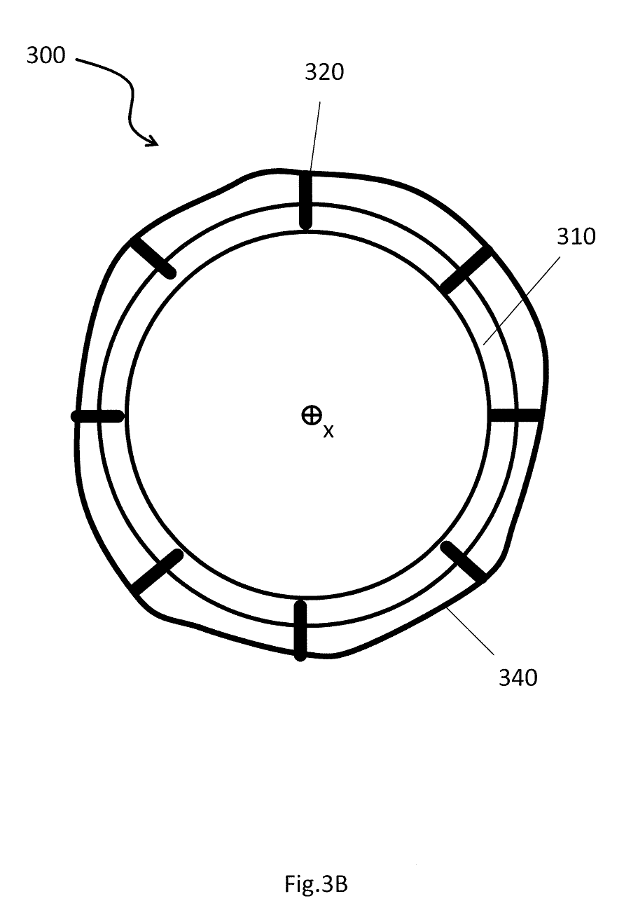

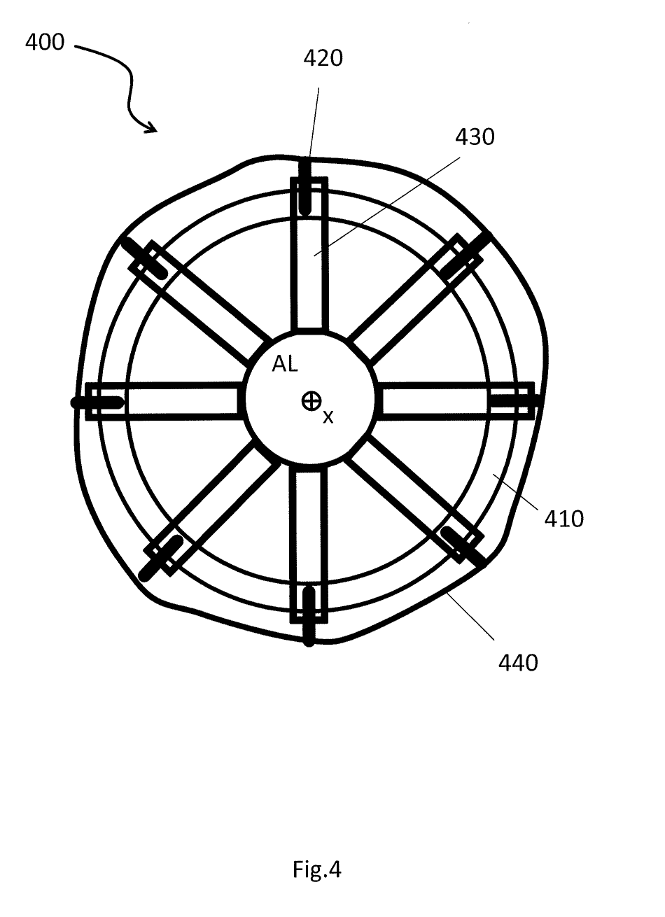

FIG. 4 schematically illustrates an arrangement according to an embodiment of the present invention with an additional collapsible tape and haptics in a rear view;

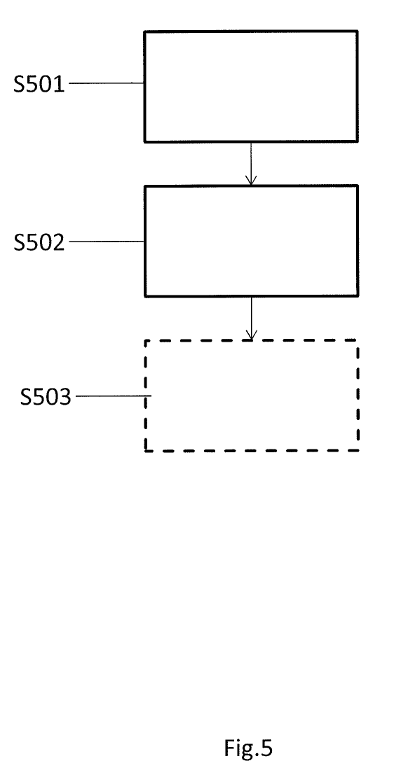

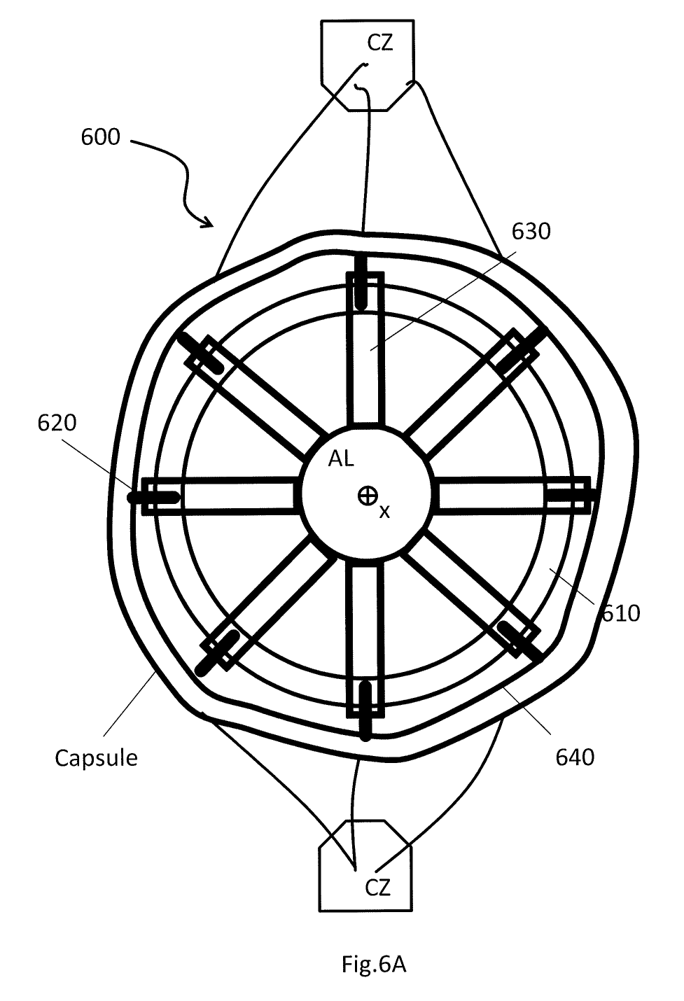

FIG. 5 schematically illustrates a surgical method according to an embodiment of the present invention;

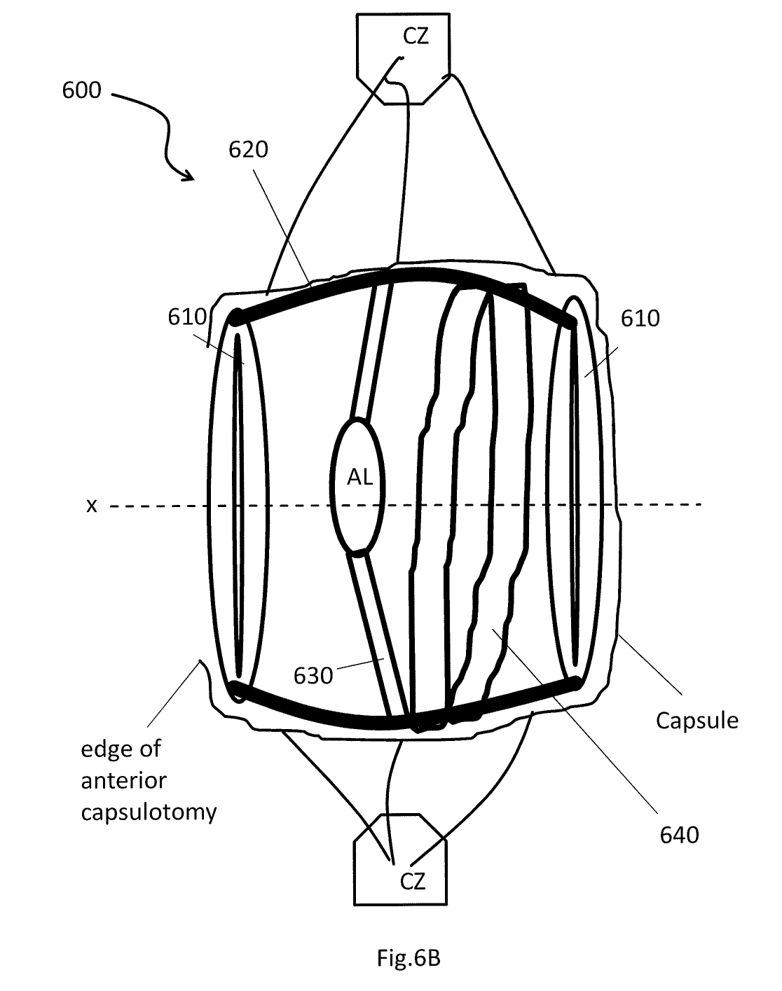

FIG. 6A schematically illustrates an arrangement according to an embodiment of the present invention inserted into an eye in a rear view; and

FIG. 6B schematically illustrates an arrangement according to an embodiment of the present invention inserted into an eye in a side view.

In the following description, for purposes of explanation and not limitation, specific details are set forth, such as specific details of the arrangement. It will be apparent to one skilled in the art that the present disclosure may be practiced in other embodiments that depart from these specific details.

FIG. 1A schematically illustrates an arrangement 100 according to an embodiment of the present invention for accommodating an artificial eye lens. The arrangement 100 comprises two ring-shaped elements 110 and a plurality of arched ribs 120. A common axis x is illustrated going through both of the ring-shaped elements 110. The ring-shaped elements and the plurality of arched ribs 120 are arranged such that the ring-shaped elements 110 are moveable in the x-direction. This leads to an accommodation of an introduced artificial lens within the space. The space is defined by the interior of the arrangement 100. When the ring-shaped elements 110 are moving outwards with respect to each other along the axis x, the plurality of arched ribs 120 are being stretched. When the two ring-shaped elements 110 are moving inwards with respect to each other along the axis x, the plurality of arched ribs 120 are being bent. Bending and stretching of the plurality of arched ribs 120 leads to the artificial lens being deformed. This kind of deformation leads to an accommodation. The deformation can be viewed as a kind of amplification of the artificial lens, which might be higher, if the artificial lens is smaller. The object is to place this arrangement within a human eye in a viewing direction which might be aligned with the illustrated axis x forming a common axis. The plurality of arched ribs 120 may be placed snugly within the capsule of a human eye that is affected by the zonules of the human eye. The capsule mentioned herein is also referred to as lens capsule in the present disclosure.

FIG. 1B schematically illustrates the arrangement 100 of FIG. 1A in a rear view. The common axis x is defined by the alignment of the two ring-shaped elements 110. The two ring-shaped elements 110 are connected via a plurality of arched ribs 120, which leads to a moveable arrangement 100. The two ring-shaped elements 110 are moveable with respect to each other along the direction given by the common axis x. In view of FIG. 1B, the plurality of arched ribs 120 may be deformed outwardly from the common axis x, or inwardly to the common axis x.

FIGS. 1C and 1D schematically illustrate an arrangement 100 according to FIGS. 1A and 1B respectively. The arrangement 100 is compressed along the common axis x. The two ring-shaped elements are moved together by a specific distance. This leads the plurality of arched ribs 120 to be bend away from the common axis x. This principle can be extended in the other direction. The plurality of arched ribs is then stretched. This correlates with the two ring-shaped elements moving away from each other along the common axis x. The ring-shaped elements 110 are held in position by the plurality of arched ribs 120. When the plurality of arched ribs 120 is unbent, the two-ring shaped elements 110 are positioned in a first position. When the plurality of arched ribs 120 is bent, the two-ring shaped elements 110 are positioned in a second position. When inserted into the human eye, the arrangement 100 is prone to the forces provided by the ciliary body via the zonules to the capsule. The accommodation is provided by the movement of the plurality of arched ribs 120 leading to an artificial lens to be deformed. This leads to the artificial lens to be stretched or compressed in order to accommodate. The two-ring shaped elements 110 provide stability for the arrangement 100. The two ring-shaped elements are coaxially aligned as illustrated. This principle can be extended by further elements to be included in the arrangement 100. Another element may be added to the arrangement 100 as shown in FIG. 2.

FIG. 2 schematically illustrates the arrangement according to FIG. 1B in a rear view along a common axis x. In the arrangement of FIG. 2, the arrangement of FIG. 1B is extended by haptics 230, which are shown as strands within the illustration. The haptics 230 are illustrated as attached to the plurality of arched ribs 220, which are themselves attached to the two ring-shaped elements 210. The haptics 230 are added to form another space in the interior of the space provided by the arrangement according to FIGS. 1A and 1B. The space is filled with an artificial lens, exemplary illustrated in FIG. 2. These haptics 230 enable the artificial lens to have an amplified or de-amplified deformation by movement of the plurality of arched ribs 220 and in connection with the two ring-shaped elements 210. This arrangement 200 being implemented into a human eye will allow the ciliary body via the zonules and capsule to transmit an even better accommodative force to the artificial lens.

FIG. 3A schematically illustrates a side view of an arrangement 300 according to an embodiment of the present invention that is based on the embodiment of FIG. 1A. The arrangement 300 comprises two ring-shaped elements 310, a plurality of arched ribs 320 and a collapsible tape 340. The collapsible tape 340 is introduced to provide a second position, which is defined by the extent of the collapsible tape 340. When the two ring-shaped elements 310 are moving with respect to one another along the x-axis, the collapsible tape 340 will assume a different shape. At a second position the collapsible tape will assume its maximum extent. This maximum extent is nearly ring-shaped and leads to the arrangement not being able to move further. This maximum extent sets the second position for the two ring-shaped elements 310. At the second position, the ring-shaped elements 310 cannot move towards each other anymore. From the second position, the ring-shaped elements 310 are only able to move away from each other along the common axis x. The underlying advantage of this collapsible tape is a predetermined distance focus provided by the length of the collapsible tape. It may be possible to set or adjust the collapsible tape, and hence distance vision, intra-operatively using refraction or wavefront techniques.

FIG. 3B schematically illustrates the arrangement 300 according to FIG. 3A from a rear view with a collapsible tape 340 shown in a position being a non-maximum extent position. When the two ring-shaped elements 310 are moving towards each other along the common axis x, the collapsible tape 340 will take on a ring-shaped form. This ring-shaped form, which might be regarded as the maximum extent, will define the second position leading to a precise distance focus.

FIG. 4 schematically shows an arrangement 400 according to an embodiment of the present invention in a rear view along a common axis x that is based on the embodiments of FIGS. 1A, 2 and/or 3A. The arrangement 400 comprises two ring-shaped elements 410, a plurality of arched ribs 420, haptics 430 and a collapsible tape 440. An artificial lens AL is schematically illustrated within the illustration held by the haptics 430, which may be strands. These strands are connected to the plurality of arched ribs 420, which are themselves connected to the two ring-shaped elements 410. The collapsible tape 440 surrounds the arrangement 400, especially the arranged plurality of arched ribs 420. The collapsible tape 440 might be arranged at a center or a vicinity of the center of the plurality of arched ribs 420. When the two ring-shaped elements are moving towards each other along the common axis x, the collapsible tape will tense and will be extended to a second position. Until the second position is reached, the plurality of arched ribs 420 will bend stronger, thereby forcing the haptics 430, which may be strands, to move outwardly away from the center of the arrangement and/or away from the common axis x. This will cause the artificial lens AL to be deformed, in this case to be extended outwardly and flattened. Thereby, the artificial lens AL will experience an amplified deformation. Thereby, the light path is changed leading to a better accommodation at distance focus being the one end of a large range of viewing distances. When the two ring-shaped elements 410 are moving away from each other along a common axis x, the plurality of arched ribs 420 will be stretched, causing the haptics 430 to move inwardly into a direction to the common axis x or the center of the arrangement 400. This causes the collapsible tape to be stretched less. A balanced situation, where the forces are stable, may be defined as a first position. The first and the second position are regarded as the beginning and end point of a range of motion of the two ring-shaped elements 410. At the first position the artificial lens AL is not deformed by outside forces being applied by the haptics 430 transmitted by the human being's zonules. When the haptics 430 are not placed at a center of the plurality of arched ribs 420, the amplification of an optical signal is performed by moving the artificial lens along the common axis x. This might even lead to a small deformation of the lens, since the movement might infer a small deformation.

FIG. 5 schematically illustrates a surgical method. The surgical method comprises introducing, in step S501 an incision into the eye. The surgical method further comprises introducing, in step S502, elements of an arrangement, as described herein, with an artificial lens into the interior of the eye. The surgical method may further comprise at least partially assembling the arrangement in the eye, in step S503. The method may be used to assemble any of the arrangements as described with respect to FIGS. 1A to 4.

FIG. 6A schematically illustrates an arrangement 600 according to an embodiment of the present invention that is arranged in an eye in a rear view. The arrangement 600 may correspond to or may be based on any of the embodiments described with respect to FIGS. 1A to 4. The ciliary zonules CZ are illustrated in FIG. 6 to hold the capsule of the natural lens that has been operated on in place. The ciliary zonules set the movement of the capsule, which fits snug around the arrangement 600 and this sets the movement of arrangement 600 and its elements 610, 620, 630 and 640. When the ciliary body contracts or relaxes, the ciliary zonules CZ follow this movement. Consequently, when the ciliary body contracts, the plurality of arched ribs 620 stretch. At the same time, the two-ring shaped elements 610 move with respect to each other so that they move away from each other. In consequence, the haptics 630 move inwardly to the center of the arrangement 600. Subsequently, the artificial lens AL is compressed by the action of the haptics 630 and also the lens may be designed like the natural human lens to be more round at rest and assume this position when the tension on it is relieved. On the other hand, when the ciliary body relaxes, there is tension on the ciliary zonules and hence on the capsule and the arrangement causing the plurality of arched ribs 620 to bend. At the same time, the two-ring shaped elements 610 move with respect to each other so that they move toward each other. In consequence, the haptics 630 move outwardly from the center of the arrangement 600. Subsequently, the artificial lens AL is stretched by the action of the haptics 630.

FIG. 6B schematically illustrates an arrangement according to an embodiment of the present invention inserted into an eye in a side view. The difference to FIG. 6A lies in the arrangement of the artificial lens AL. The artificial lens AL is movably arranged within the arrangement 600. The haptics 630 are adapted to move the artificial lens AL along the axis x. This is due to their oblique alignment. In consequence, the artificial lens AL will move along the axis x, when forced by the haptics 630. This is another way of accommodating to specific viewing distances. The artificial eye lens moves to the left in this embodiment, when the two ring-shaped elements 610 move away from each other, since the plurality of arched ribs 620 are stretched. Further the haptics 630 move inwards in a direction to the x axis. In consequence, the artificial lens moves to left in FIG. 6B along the x axis. The artificial lens AL moves to the right in FIG. 6B, when the two ring-shaped elements 610 move towards each other. The plurality of arched ribs 620 then bend and force the haptics 630 to move in a direction away from the x axis. This causes the artificial lens AL to move to the right in FIG. 6B along the x axis. Further, in FIG. 6B, the collapsible tape 640 is shown to be arranged such that a movement of the arrangement is limited/restricted. This has the advantage to optimally adjust the arrangement in its movability and/or adjustment of distance focus.

Another potential use of this arrangement is that as it spatially occupies more or less the fill of the bag of the natural human lens and that the suspended lens is predictably placed in the arrangement the position of the lens in the optical system of the eye is quite predictable. This is advantageous in calculating the focus of the lens to coincide with e.g. emmetropia (distance focus) and could be useful even for fixed focus or multifocal lenses, especially if assembled in the eye through a small incision.

1. An arrangement for an intraocular lens, the arrangement comprising:
at least two ring-shaped elements
a plurality of arched ribs connecting the two ring-shaped elements, the ring-shaped elements and the plurality of arched ribs forming a space for housing the intraocular lens and the ring-shaped elements being moveable, with respect to each other, along a common axis; and
a collapsible tape adapted and arranged to link the arched ribs and define a maximum range of deformation of the arrangement.

2. The arrangement of claim 1, wherein the ring-shaped elements are arranged in parallel to each other, and/or the arched ribs are flexible and/or stretchable.

3. The arrangement of claim 1, the arrangement comprising haptics that are configured and arranged to allow the intraocular lens to be connected thereto within the space,
wherein the haptics comprise one or more legs or strands respectively protruding from at least one of the plurality of arched ribs into the interior of the space.

4. The arrangement of claim 1, wherein the ring-shaped elements are moveable, with respect to each other, along the common axis at least between a first position and second position, wherein in the first position, the arched ribs are more flattened than in the second position.

5. The arrangement of claim 4, wherein, in the first position, the distance between the two ring-shaped elements along the common axis is larger than in the second position.

6. The arrangement of claim 1, the arrangement further comprising the intraocular lens.

7. The arrangement of claim 6, wherein the arrangement is configured to hold, move or deform the intraocular lens.

8. The arrangement of claim 6, wherein the arrangement is adapted to adjust a distance focus of the intraocular lens,
wherein the arrangement is adapted to adjust the distance focus of the intraocular lens by deforming the intraocular lens, or by moving the intraocular lens along the common axis provided by movement of the plurality of arched ribs.

9. The arrangement of claim 1, wherein the second position is set by an extent of the collapsible tape, wherein in the second position the collapsible tape has a ring-shaped form.

10. The arrangement of claim 1, wherein the collapsible tape is arranged on the center or a vicinity of the center of each of the arched ribs.

11-12. (canceled)
